(19) United States
(12) Patent Application Publication
(10) Pub. No.: US 2017/0173364 A1
(43) Pub. Date: Jun. 22, 2017

(54) NANOPARTICLE-MEDIATED ABLATION OF GLIOBLASTOMA AND OF OTHER MALIGNANCIES

(71) Applicant: NANOPROBES, INC., Yaphank, NY (US)

(72) Inventors: James Frederick HAINFELD, Shoreham, NY (US); Daniel Nathan SLATKIN, Essex, CT (US)

(73) Assignee: NANOPROBES, INC., Yaphank, NY (US)

(21) Appl. No.: 15/301,814

(22) PCT Filed: Apr. 27, 2015
(86) PCT No.: PCT/US15/27737

§ 371 Date: Oct. 4, 2016

Related U.S. Application Data
(60) Provisional application No. 61/995,947, filed on Apr. 25, 2014.

(51) Int. Cl. A61N 5/10; A61N 7/00; A61K 41/00; A61B 18/18

(52) U.S. Cl. CPC A61N 5/10; A61B 18/18; A61N 7/00; A61K 41/0038; A61K 41/0095; A61N 2005/1098

(57) ABSTRACT

We disclose methods of treating cranial neoplasm using nanoparticles that are administered to the patient's brain. Our nanoparticles are novel, designed and synthesized explicitly to ensure their preferential accumulation in all parts of most brains that have remained with latent glioblastoma cells immediately after any primary neurosurgical debulking, and little or no accumulation in other parts of the brain.

The present application claims priority of U.S. Provisional Application Ser. No. 61/995,947, filed Apr. 25, 2014, herein incorporated by reference in its entirety. The present invention is directed to nanoparticles and methods us thereof in treating cranial neoplasms.

### FIELD OF THE INVENTION

### Background

Glioblastoma multiforme (“glioblastoma”) is the principal primary brain neoplasm (tumor) of humans, lethal to approximately 80% of humans who die of any malignancy arising in the brain. In the United States, it kills nearly 10,000 persons per year. Glioblastoma is generally unifocal and does not usually metastasize. There exist various powerful therapeutic methods for killing other malignant cells and non-metastasizing unifocal malignant neoplasms using chemicals, surgical extirpation, radiation, and/or other techniques. The common impediment to cures of glioblastoma by any or all such techniques used to date clinically is the neurological vulnerability to such techniques of the brain that harbors glioblastoma.

Typically a glioblastoma has an abnormal accumulation of fluid and accompanying swelling (edema) associated therewith surrounding or adjacent the tumor. A cerebral edema is excess accumulation of fluid in the intracellular or extracellular spaces of the brain. One of the most common complications of brain tumor growth is the resulting peritumoral edema (hereafter referred to simply as edema; also called oedema). Edema is a major cause of neurological deficits, an independent prognostic factor for overall survival, and often the condition which ultimately causes death in high grade brain tumor patients. The causes of peritumoral edema are not fully understood, but it is believed that edema forms as a result of excess fluid buildup in the extravascular space surrounding the tumor and an inability of the brain to clear this fluid due to the injured blood brain barrier.

The grade of edema is closely related to the degree of malignancy in all brain tumors as well as the location of the tumor within the brain. In glioblastoma, nearly all progressing tumors show a large degree of edema. Because of the demonstrated role of inflammatory cytokines in edema production, especially the specific role of cyclooxygenase-2 (COX-2), a wide variety of substances are available which may provide relief against edema. The gold standard treatment against edema however, is a corticosteroid called dexamethasone (Decadron®). Despite this however, dexamethasone has significant adverse side-effects which should encourage patients to try other edema treatment options.

Corticosteroids like Decadron® are powerful and fast-acting, with dose-dependent benefits and side-effects, especially when used in large amounts over long periods. Decadron® use is often necessary as a temporary, precautionary measure after surgery, but patients should generally try to wean off of Decadron® if possible. Corticosteroid dosages must be tapered slowly over time to let the body's natural adrenal functions take effect and to prevent severe withdrawal-like symptoms.

A major challenge in treating a glioblastoma and the peritumor edema associated therewith, is that there is no reliable way to predict the extent to which brain edema must be controlled after a procedure (both surgical or non-surgical) which removes or reduces the tumor. Physicians and patients are often left with the precarious goal of using trial and error methods to determine how best to treat the area in association with any procedure.

Much of this is documented in a German medical journal by Hartmann et al. in 1998, where seventy-nine essentially sequential patients with histologically verified cerebral malignant gliomas were studied by magnetic resonance imaging (MRI) before, within 3 days following, and every 2-3 months after their initial glioma-debulking surgery. The median follow-up was 11 months, during which time forty-seven of those seventy-nine patients showed initial tumor recurrence by clinical magnetic resonance imaging (MRI). Authors Hartmann et al. in 1998 correlated the configurations of the initial vasogenic edema on T2-weighted MRIs with tumor-regrowth patterns on contrast-enhanced T1-weighted MRIs. 35/47 tumor regrowths began within the initial (predebulking) edema configurations; 11/47 occurred within the zones of the predebulked gliomas; tumor recurrence was multifocal in only one of those forty-seven patients. Thus, we assume (not improbably) that the recurrent tumor did not originate from new clonogenic glioma tumor cells that arose de novo in the brain but from the original clonogenic glioma tumor cells, whereby 11/47 of those patients had recurrences from zones of glioma noted on MRI but not removed by the neurosurgeon during the initial debulking; 35/47 of them had recurrences from individual or clumps of clonogenic glioma cells (“guerrilla” cells) that had migrated or had drifted to somewhere within the peritumor edema fluid from the margin of the tumor prior to its removal by the neurosurgeon but had not moved beyond the zone of peritumor edema; but only 1/47 patients had recurrences of glioma from clonogenic glioma tumor cells that had drifted outside the zone of peritumor edema.

There is therefore a desire for novel therapeutic methods of treating neoplasms, particularly in the cranial area, which focus on providing an aid to treading the general area surrounding the neoplasm, including the peritumor edema area.

### SUMMARY

The present invention is a method of enhancing a cranial neoplasm procedure, and nanoparticles used therein. The method includes i) inserting nanoparticles with a mean particle diameter of 1 nanometer to 1,000 nanometers into an area of the neoplasm, and ii) applying energy in the form of radiation to the nanoparticles to treat the neoplasm area. The present invention also provides for the use of the nanoparticles in the disclosed methods.

In an embodiment, the nanoparticles migrate to areas surrounding the neoplasm prior to applying the radiation. In another embodiment, the areas surrounding the neoplasm include areas of edema. In a further embodiment, the areas of edema further include cancerous tissue or secondary tumors associated with the neoplasm. In yet another embodiment, the cranial neoplasm procedure is a surgical procedure which reduces or extirpates the neoplasm. In another embodiment it is a minimally invasive procedure with reduces or extirpates the neoplasm.

In an embodiment the cranial neoplasm is a glioblastoma. In another embodiment, the nanoparticles are introduced into the neoplasm area in a reservoir. In an embodiment, the reservoir is a sponge, solution, suspension, or gel. In an embodiment, the mean particle diameter of the nanoparticles is between about 1 and about 200 nm. In another embodiment, the mean particle diameter of the nanoparticles is between about 10 and about 100.

In an embodiment, the radiation is selected from the group of x-rays, alternating magnetic fields, static magnetic fields, electrons, beta and gamma rays, alpha particles, protons, muons, pions, positrons, antiprotons, carbon ions, infrared radiation, microwaves, neutrons, radiofrequency or other-frequency electromagnetic and/or synchrotron/electrical and/or centrifugal/explosive waves/photons, and/or ultrasonic waves through any solid, semi-solid, gelatinous, liquid and/or gaseous medium. In a preferred embodiment, the radiation is in the form of x-ray radiation.

In an embodiment, the nanoparticles are comprised of a material selected from the group of iodine, gold, bismuth, platinum, hafnium, holmium, iridium, europium, gadolinium, uranium, or tungsten. In an embodiment, the nanoparticles are comprised of a material selected from the group of iodine, gold, bismuth, hafnium, holmium, gadolinium, or tungsten.

For a better understanding of the present invention, together with other and further objects and advantages, reference is made to the following detailed description, taken in conjunction with the accompanying examples, and the scope of the invention will be pointed out in the appended claims. The following detailed description is not intended to restrict the scope of the invention by the advantages wet forth above.

### BRIEF DESCRIPTION OF THE DRAWINGS

FIG. 1 diagrammatically depicts a brain tumor with a central tumor mass and radiating regions of peritumor edema.

FIG. 2 shows an enlargement of the tumor showing tumor cells in the peritumor edema and now surrounded by the nanoparticles instilled as shown in FIG. 1.

### DETAILED DESCRIPTION

We disclose nanoparticles that are administered to the patient's (or animal's) brain directly and/or via the blood and/or the cerebrospinal fluid. Our nanoparticles are novel, designed and synthesized explicitly to ensure their preferential accumulation in all parts of most brains that have remained with latent glioblastoma cells immediately after any primary neurosurgical debulking, and little or no accumulation in other parts of the brain. This application is directed to both the invention of those nanoparticles as described herein, and their clinical use in human and/or veterinary medicine for therapy of glioblastoma in humans and/or therapy of any other human neoplasm and/or therapy of any neoplasm in any animal.

Specifically, we propose that biocompatible, preferably but not necessarily biodegradable sponges or other reservoirs (including solutions, suspensions, or gels) of therapeutically binary nanoparticles be inserted into the zone of cancer invasion or into the tissue space or spaces that remain after visibly complete or visibly partial surgical or other method of reduction or extirpation of a cancer. The nanoparticles may also be delivered by slow injection, commonly referred to as ‘convection enhanced delivery.’

As used herein the term “therapeutically binary,” refers to nanoparticles which do not attain their optimal therapeutic efficacies unless and until they are perturbed or modified during or after their insertion by another physical or chemical process. Although tumor therapists have taught and/or have implemented placement of various reservoirs of cancer-therapeutic substances and nanoparticles at diverse locations in the bodies of cancer patients, none has proposed designing and synthesizing therapeutically binary nanoparticles in such a specific manner as to ensure, not only that they will be optimally efficacious only after perturbation or modification by another physical or chemical process, but that they will have weights, sizes, compositions, biocompatible coatings, optimal diffusion characteristics in peritumor edematous tissues, optimal resistance to leakage away from the zone of peritumor edema, and such other designs as to endow the nanoparticles with attributes optimized for least toxicity to vital normal tissues and most toxicity to the cancer so treated.

Clinical evidence we have related above indicates not only that clonogenic glioma tumor cells are known to lack a general propensity to metastasize beyond the central nervous system, but they also generally lack the propensity to migrate beyond the zone of brain edema induced in their immediate surroundings within the central nervous system, the therapeutic implications of which have not been disclosed or applied heretofore in conjunction with appropriately designed, synthesized, and therapeutically effective nanoparticles. Not only does our analysis explain why malignant gliomas are so difficult to mitigate by knife surgery, much less to cure, but also presents a novel, simple, to date, surprisingly untaught and unexploited nanoparticle-mediated method of therapy to supplement blunt tumor knife-debulking. Although Hippocrates taught that drastic remedies are needed for devastating diseases, the present invention of biocompatible-nanoparticle-mediated therapy reveals additional remedies for devastating diseases that are far from drastic: they convey no significant added harm to patients so devastatingly afflicted we can anticipate.

We hereby disclose a method for preferential nanoparticle-enhanced treatment of the peritumor edema zone in and around brain tissues after a surgeon debulks a brain tumor. Unlike the existing practice with brain tumors, closing the surgical wounds then discharging the patient from the operating theater to recover from anesthesia, in our method the surgeon inserts one or more prepared, nanoparticle-suffused, sterile or microbiologically, virologically, genetically, hormonally, immunologically and/or otherwise therapeutically doped, biocompatible reservoirs such as osmotic pumps, wafers, sponges, and/or any other kind of porous gels, solutions, suspensions or solids into the wounds before proceeding with the surgical wounds' closures or provides a delivery tube or stereotactic path by which such instillations can be made. The nanoparticles are designed and synthesized in such a way as to diffuse spontaneously throughout all or most of the peritumor edema zone from their reservoirs, no matter how tortuous and extensive that zone may be in any particular patient, but to be inhibited if not prevented and/or stopped from diffusing beyond peritumor edema zones. Apart from the additional short time needed for the surgeon to insert said prepared reservoirs into the spaces created by the debulking procedure, standard surgical practices, recovery from anesthesia, and postsurgical wound healing are as they would be without prior insertion of the nanoparticle reservoir.

This method could be adapted to assist in the therapy of other malignancies that are first extirpated surgically, for example, certain breast cancers, ovarian cancers, soft-tissue sarcomas, gallbladder cancers, urinary bladder cancers, cerebrospinal metastases, and salivary gland cancers, for which post-operative radiotherapy would routinely be prescribed to prevent, inhibit and/or delay recrudescence of the malignancy.

The therapeutic merits of this novel procedure are: 1) unlike existing methods, whereby reservoirs themselves contain therapeutically bioactive anti-cancer substances that diffuse slowly or quickly into the peritumor edema zone, the reservoirs described here are not bioactive or therapeutic, but contain nanoparticles that diffuse into the far reaches of the possibly tortuous peritumor edema zone before they are activated, after which most therapeutic effects of them and/or of the substances weakly or strongly linked to them are exerted on their surrounding cells and tissues; 2) the concentrations of said nanoparticles in the near and far reaches of the peritumor edema zones will not be substantially reduced by minimal diffusion beyond those zones before their therapeutic effects are exerted on cells and tissues within the peritumor edema zone; 3) most important is that residual malignant stem cells in the peritumor edema zone will be accessible to lethal effects of those nanoparticles, no matter how extensive or tortuous said zone may be a consequence of this novel process not disclosed or implemented heretofore.

It is known, for example, that the large majority of glioblastoma tumors relentlessly recur after even wide surgical debulking, however sophisticated the surgical methods to safely extirpate the malignancy from the brain may be, although it is also known that the large majority of such recurrences begin in some zone of peritumor edema extant before or shortly after the original debulking. The present invention is based on the premise that the glioblastoma recurrence begins from one or more of such residual malignant stem cells active or dormant somewhere hidden in the extensive but rather well demarcated zone of peritumor edema. Although many others have expressed similar surmises, none has proposed a method to deploy lethal nanoparticles in the same peritumor edema zones in a manner that enables the tumor stem cells hidden therein to be ablated by radiation. Evidently, those stem cells cannot be eradicated by existing methods of therapy, because radiotoxicity to normal nearby tissues would be contraindicative. Hence the procedure we describe is original, since conceptually and fundamentally it enables lethal therapy to be exerted on each individual malignant stem cell lurking in that zone, wherever it may be.

An example of the application of this invention comprises, but is not limited to so using nanoparticles containing heavy atoms such as iodine, gold, bismuth, platinum, hafnium, holmium, iridium, europium, gadolinium, uranium, or tungsten. In that example, the zone of brain surmised or imaged that contains all or nearly all of the zone of peritumor edema can be irradiated by orthovoltage X-rays and/or megavoltage X-rays up to maximally radiotoxically tolerated doses: although these doses are long known to be insufficient in themselves to eliminate post-debulking residual glioblastoma stem cells, secondary electrons emanating from those heavy atoms can be lethal. In the particular example of gold nanoparticles, the dose delivered to a 30-micrometer-wide shell around the gold can be double the general dose, and the high spatial density of the free radicals so induced can double their relative biological effectiveness (Seidenbusch M, Harder D, Regulla D. Systematic survey of the dose enhancement in tissue-equivalent materials facing medium-and high-Z backscatterers exposed to X-rays with energies from 5 to 250 keV. Radiat Environ Biophys (published on line, Mar. 15, 2014: DOI 10.1007/s00411-014-0524-y), causing a safe radiosurgical dose, for example, 15 gray, to be, in effect, quadrupled to 60 gray-equivalent to glioblastoma stem cells adjacent to or contiguous with any gold-containing nanoparticle in a 15-gray radiation field. By stereotactic techniques well known in the arts of radiosurgery and brachytherapy, when such irradiated fields zones do not in total occupy an excessively large proportion of the intracranial volume, and that by single radiation dose or dose fractionated temporally, the macroscopic dose of X-rays might be reasonably, if not safely doubled to 30 Gy, for example in a patient bearing an inevitably, inexorably, and rather quickly fatal malignancy such as glioblastoma, exerting the equivalent of 120 gray on residual glioblastoma stem cells, which should be sufficient to clonogenically disable every such cell, irrespective of its particular genetic makeup.

In other embodiments, the nanoparticle can be a polymer, a biodegradable polymer, a polymer containing poly (lactic-co-glycolic acid) (PLGA), polyethylene glycol, a liposome, a heat-sensitive liposome, a boron-10- and/or gadolinium- and/or magnetite-containing nanoparticle, a nanoparticle with a silicon oxide layer, a nanoshell, a rod, a sphere, an oxidized iron nanoparticle, a tungsten- or bismuth-containing nanoparticle. Any nanoparticle or object up to 1,000 nm in least diameter (capable of being stored in, then steadily released from a biocompatible reservoir) fully or partially enveloped by or linked to a shell, polymer, emulsion, containing/surrounding/comprising elements or compounds that can be activated to biolethality spontaneously or by the environment around the tumor zone and/or deliberately at a time chosen by a cancer therapist, and/or by any caretaker, and/or by the patient himself/herself, by externally or locally applied energy or radiation.

In other embodiments, the nanoparticle can be coated with a shell consisting of, but not limited to: polyethylene glycol (PEG), polyvinyl alcohol, sugars, dextrans, reactive moieties, hyaluronan, silanes and other organic molecules. Nanoparticles useful in the present application include, but are not limited to those disclosed in U.S. Pat. Nos. 5,521,289; 6,121,425; 6,369,206; 6,955,639; 7,367,934; 7,906,147; and 8,033,977; each of which is herein incorporated by reference in their entirety.

The methods disclosed herein could also be applicable to malignancies without being associated with their initial or subsequent surgical reduction/resection, especially in cases that would require difficult and/or intricate, possibly dangerously or inconveniently prolonged surgical interventions due to their locations or complexities, or due to the fragile health of a patient. In such cases, the reservoir or multiple reservoirs would be instilled/inserted around the tumor from which the nanoparticles would suffuse the peritumor edema. Since the peritumor edema exists as a regularly or irregularly shaped, continuous or almost continuous shell around the tumor and/or the site of tumor surgery, subsequent irradiation, killing adjacent cells and blood vessels at the tumor boundary, would cut off a non-metastasized tumor from its blood and nutrient supply, almost certainly resulting in some tumor suppression and, possibly, tumor demise.

Our invention, in summary, is the design, synthesis, and clinical deployment and use of inert but potentially bioactive nanoparticles that have these attributes: 1) diffuse freely into peritumor edema tissue from a reservoir of them placed in a region of peritumor edema or in a tumor bed immediately after macroscopically visible and/or safely permissible surgical reduction or extirpation of a malignant tumor; 2) minimal diffusivity (before external activation) into tissues beyond the margins of the peritumor edema zone other than into the lymphatics draining the peritumor edema tissue; 3) following tumor cells to, and enveloping tumor cells in regional lymph nodes, potential sites of metastases; 4) clonogenically or immediately biolethal or biodisabling when sufficient numbers of them are activated while in microscopic apposition to, in close proximity with, juxtaposed to, and/or within guerilla cells; 5) nonlethal if undisturbed; 6) nonpoisonous if undisturbed; 7) pharmacologically tolerable.

Once the nanoparticles are instilled and suffuse the peritumor edema, external or locally applied energy is applied that interacts with the nanoparticles resulting in severe damage or death to tumor cells in the peritumor edema. The applied energy can be in the form of, but not limited to: X-rays, alternating magnetic fields, static magnetic fields, electrons, beta and gamma rays, alpha particles, protons, muons, pions, positrons, antiprotons, carbon ions, infrared radiation, microwaves, neutrons, radiofrequency or other-frequency electromagnetic and/or synchrotron/electrical and/or centrifugal/explosive waves/photons, and/or ultrasonic waves through any solid, semi-solid, gelatinous, liquid and/or gaseous medium.

Another case disclosed is externally applied energy that activates the particles in the peritumor edema to release a drug; e.g., ultrasound effect on perfluorocarbon constructs carrying a drug; or heating a heat-sensitive liposome containing a drug that is released upon heating; mechanical shock-wave triggering a release of an otherwise encapsulated, cancer-suppressive drug.

In other embodiments, the nanoparticle can have attached a surface targeting moiety that directs the particle to the tumor cells, to enhance binding to them, or to enhance internalization, or to enhance targeting to cellular enzymes, DNA, RNA, proteins, lipids, or carbohydrates. The targeting moiety can be selected from the group, but not limited to the group of: antibodies, antibody fragments, cell ligands, aptamers, DNA, RNA, drugs, compounds that enhance targeting, and other groups or materials that enhance targeting.

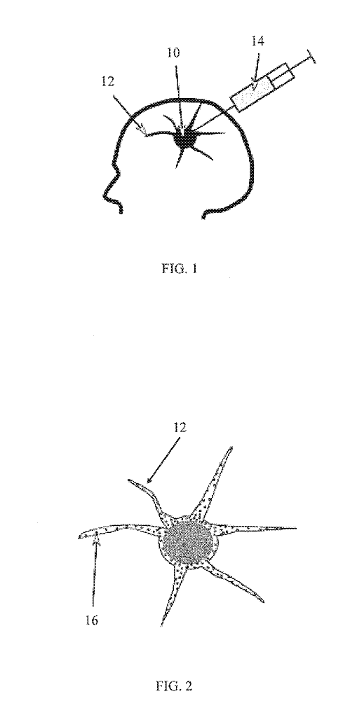

With reference now to the Figures, FIG. 1 shows a cranial neoplasm 10, or tumor. The neoplasm 10 has pertiumor edema 12 associated therewith. Nanoparticles 14 can be injected into the general area of neoplasm 10, and diffuse to areas of the edema 12. FIG. 2 shows peritumor edema 12 with tumor cells 16 dispersed throughout.

The inventors of the present invention have designed, synthesized, and successfully tested in mice biocompatible gold nanoparticles which, upon intravenous injection extravasated from tumor vasculature in orthotopic gliomas in mice and spread into the zone of peritumor edema, not necessarily beyond the peritumor edema zone, to locations in proximity to clonogenic malignant stem cells (i.e., in proximity to “guerrilla” cells) in such high concentrations that after an X-ray dose of 30 Gy achieved a permanent cure of a substantial fraction of the malignant tumors in those mice that otherwise would have progressed to kill their hosts. All mice that did not receive the gold nanoparticles, but had gliomas and either did or did not receive the 30 Gy X-ray dose died of tumor overgrowth. The particular nanoparticles and externally or locally applied activating energies that may be used in this invention where the particles are intended to be introduced locally to the tumor include: 1) gold nanoparticles that were subsequently injected then had spontaneously migrated in and around the tumor then were excited by otherwise poorly effective doses of externally applied orthovoltage X-rays or megavoltage X-rays to emit secondary radiations that clonogenically disabled guerrilla cells adjacent to them, 2) gold nanoparticles that had been injected then had spontaneously migrated in and around the tumor then were excited by otherwise poorly effective doses of externally or locally applied infrared radiation to become so hot that otherwise therapeutically intractable guerrilla cells adjacent to them had become clonogenically disabled, and 3) iron nanoparticles that had been injected then had spontaneously migrated in and around the tumor then were excited by otherwise poorly effective alternating external magnetic fields to become so hot that otherwise therapeutically intractable guerrilla cells adjacent to them had become clonogenically disabled; 4) particles containing boron-10 activated by neutrons, 5) particles heated by radiofrequency electromagnetic photons/waves, 6) particles activated by ultrasonic waves in liquids and/or gases and/or solids, and/or gels (In summary, in any sound-transmitting material), 7) particles activated by “microwave” electromagnetic energy, 8) particles activated by protons or carbon ions, 9) particles that are heated combined with orthovoltage X-rays or megavoltage X-rays.

The common denominator of these successful treatments of experimental malignant tumors is that the nanoparticles we propose, unlike other substances used in the existing arts of clinical oncology, are not free to diffuse as are most if not all substances injected to inhibit tumor growth, but are restricted in their propensities to migrate far from their positions of injection—except within the zones of tissue edema that generally surround and extends into and between non-edematous nearby normal tissues, beyond the discernible edges of a locally growing malignant tumor. These zones are precisely where clonogenic tumor cells and microscopic clumps of same are likely to migrate, i.e., beyond the vision (enhanced by noninvasive imaging or not) of the surgeon and/or safe reach of the tumor-extirpating surgeon's knife; beyond the safe reach of non-binary radiotherapies and of non-binary brachytherapies; beyond the safe reach of systemically and locally applied chemotherapeutic and immunotherapeutic agents. Moreover, were any techniques found previously or in the future beyond the scope of this invention that could also safely reduce guerilla cells in zones of peritumor edema, the presence of pharmacologically and radiologically inert nanoparticles of our invention should be unlikely to impede the therapeutic benefit or increase the risk of other modalities prior to the activation to biolethality of the nanoparticles of our invention.

### EXAMPLES

The present invention is further exemplified, but not limited, by the following representative examples, which are intended to illustrate the invention and are not to be construed as being limitations thereto.

### Example 1—MEXRT

The inventors of the present invention propose prognoses for about three-quarters of glioblastoma patients should be improved considerably were a biocompatible sponge soaked with ≈15-500 nm-diameter, stealth-coated gold particles inserted in the debulked tumor bed then that bed plus radiating peritumor edema irradiated radiosurgically with orthovoltage X-rays several days after debulking to at least ≈20 Gy-equivalent, administered from each of several different directions toward a wide zone of the ipsilateral cerebral hemisphere. The nominal macroscopic dose to the residual tumor bed need only be ≈20 Gy (e.g., ≈4 Gy from each of four converging directions), but the microscopic dose to each guerrilla cell should be roughly fourfold higher, ≈80 Gy-equivalent, twofold greater than 20 Gy on account of the doubling from 1 to 2 of the relative biological effectiveness (RBE) of the dense shower of electrons within ≈30 micrometers of each pericellular accumulation of gold nanoparticles times twofold greater than 20 Gy on account of the doubling of the physical absorbed radiation dose within ≈30 micrometers of each pericellular accumulation of gold nanoparticles in the orthovoltage X-ray field of exposure. Since one-fraction radiosurgical doses in relatively well-demarcated, neurologically non-eloquent zones of the brain can be at least 20 Gy, the radiophysical 20 Gy dose might be increased during metal-enhanced X-ray therapy [MEXRT] to 80 Gy-equivalent, perhaps higher, within the zone of peritumor edema, without adding unacceptable radiotoxic radiosurgical injury to the already naturally and neurosurgically injured glioblastoma-bearing brain.

### Example 2—MEPT

The present invention also provides for the use of gold nanoparticles to enhance proton therapy (metal-enhanced proton therapy; MEPT) by proton irradiation of lethal effects from nanoparticles. Proton therapy to preferentially irradiate the gold-nanoparticle-suffused peritumor edema zone, wherever feasible, would offer the advantage of better conformation with peritumor edema zones in patients having many finger-like configurations rather than one ovoid configuration of peritumor edema between the macroscopically identified tumor and non-edematous normal tissues.

### Example 3—NCT

B-10 NCT or BNCT/Gd NCT or GdNCT/U-235 NCT or UNCT/Li-6 NCT or LiNCT. In the field of neutron-capture therapies (NCTs), boron neutron-capture therapy (B-10 NCT or BNCT) stands out. In another example of the hitherto untaught applications of our invention, any of the boron-containing substances hitherto used or planned to be used for BNCT could be advantageously attached or otherwise bound to the kind or kinds of nanoparticles described above or use of boron-containing nanoparticles which are then caused to suffuse a biodegradable sponge or other biocompatible reservoir that is placed into the bed of a tumor after knife-extirpation by a surgeon of all or part of said tumor prior to exposure to thermal and/or epithermal neutrons.

Thus while there have been described what are presently believed to be preferred embodiments of the invention, those skilled in the art will realize that changes and modifications may be made thereto without departing from the spirit of the invention, and it is intended to claim all such changes and modifications as fall within the true scope of the invention.

What is claimed is:

1. A method of enhancing a cranial neoplasm procedure comprising:
i) Inserting nanoparticles with a mean particle diameter of about 1 nanometer to about 1,000 nanometers into an area of said neoplasm;
ii) Applying energy in the form of radiation to said nanoparticle to treat said neoplasm area.

2. The method of claim 1 wherein said nanoparticles migrate to areas surrounding said neoplasm prior to applying said radiation.

3. The method of claim 2 wherein said areas surrounding said neoplasm include areas of edema.

4. The method of claim 3 wherein said areas of edema further include cancerous tissue or secondary tumors associated with said neoplasm.

5. The method of claim 1 wherein said cranial neoplasm procedure is a surgical procedure which reduces or extirpates said neoplasm.

6. The method of claim 5 wherein said cranial neoplasm is a glioblastoma.

7. The method of claim 1 wherein said nanoparticles are introduced into said neoplasm area in a reservoir.

8. The method of claim 7 wherein said reservoir is a sponge, solution, suspension, or gel.

9. The method of claim 1 wherein said mean particle diameter of said nanoparticles is between about 1 nm and about 200 nm.

10. The method of claim 9 wherein said mean particle diameter of said nanoparticles is between about 10 nm and about 100.

11. The method of claim 1 wherein said radiation is selected from the group of x-rays, alternating magnetic fields, static magnetic fields, electrons, beta and gamma rays, alpha particles, protons, muons, pions, positrons, antiprotons, carbon ions, infrared radiation, microwaves, neutrons, radiofrequency or other-frequency electromagnetic and/or synchrotron/electrical and/or centrifugal/explosive waves/photons, and/or ultrasonic waves through any solid, semi-solid, gelatinous, liquid and/or gaseous medium.

12. The method of claim 11 wherein said radiation is in the form of x-ray radiation.

13. The method of claim 1 wherein said nanoparticles are comprised of a material selected from the group of iodine, gold, bismuth, platinum, hafnium, holmium, iridium, europium, gadolinium, uranium, or tungsten.

14. The method of claim 13 wherein said nanoparticles are comprised of a material selected from the group of iodine, gold, bismuth, hafnium, holmium, gadolinium, or tungsten.
